(19) United States
(12) Patent Application Publication
(10) Pub. No.: US 2020/0402675 A1
(43) Pub. Date: Dec. 24, 2020

(54) REMOTE HEALTHCARE DELIVERY DEVICES, SYSTEMS AND METHODS

(71) Applicant: WhenMed VC LLC, San Diego, CA (US)

(72) Inventors: James J. Macoviak, Conyngham, PA (US); John A. Macoviak, San Diego, CA (US); Richard L. Macoviak, Weatherly, CA (US)

(21) Appl. No.: 16/921,145

(22) Filed: Jul. 6, 2020

Related U.S. Application Data
(63) Continuation of application No. 15/908,645, filed on Feb. 28, 2018.
Application No. 15/908,645 is a continuation of application No. PCT/US2016/050273, filed on Sep. 2, 2016.
(60) Provisional application No. 62/214,146, filed on Sep. 3, 2015.

(51) Int. Cl. G16H 80/00; G06Q 50/22; G16H 50/30; G16H 10/65

(52) U.S. Cl. CPC G16H 80/00; G06Q 50/22; G06K 7/0013; G16H 10/65; G16H 50/30

(57) ABSTRACT

Described herein are devices, systems, and methods for providing remote healthcare. In one or more embodiments, the devices, systems, and methods described herein are configured to provide a telemedical encounter between a healthcare provider and a subject.

### CROSS-REFERENCE TO RELATED APPLICATIONS

This is a continuation application of, and which claims the benefit of the filing date and disclosure of, U.S. patent application Ser. No. 15/908,645, filed on Feb. 28, 2018, entitled “Remote Healthcare Delivery Devices, Systems, and Methods,” which is a continuation of and claims the benefit of the filing date of International Application PCT/US2016/050273, filed Sep. 2, 2016, which is based on and claims the benefit of the filing date of U.S. Provisional Patent Application No. 62/214,146, filed on Sep. 3, 2015, the contents of each of which are incorporated by reference herein.

### BACKGROUND OF THE INVENTION

A wide variety of circumstances result in inadequate access to healthcare for many subjects and families. Some lack adequate access because they live in isolated, rural, or other governmentally designated underserved areas. Some lack adequate access because they are uninsured or underinsured. Others live in developing countries where medical training and infrastructure is yet to be developed. Circumstances render some subjects without adequate access to healthcare in natural and manmade disaster areas and battlefields.

Moreover, the cost of providing adequate healthcare is rising. While more money is spent on health care per person in the U.S. than in any other nation in the world, in 2009, the U.S. Census Bureau reported that 16.7% of the population was uninsured. Current estimates put U.S. health care spending at approximately 16% of GDP. Growth in healthcare spending is projected to average 6.7% annually over the period 2007 through 2017. High healthcare costs also affect subjects. A 2007 study found that 62.1% of filers for bankruptcy cited high medical expenses as a contributing factor.

### BRIEF SUMMARY OF THE INVENTION

Described herein are devices, systems, and methods for providing remote healthcare. In one or more embodiments, the devices, systems, and methods described herein are configured to provide a telemedical encounter between a healthcare provider and a subject.

In one or more embodiments, the devices, systems, and methods described herein comprise biosensors that are configured to monitor biometric parameters of a subject. In one or more embodiments, one or more biosensors are configured to sense an electrocardiogram. In one or more embodiments, a biosensor is configured to sense a temperature of a subject. In one or more embodiments, a biosensor is configured to sense a blood pressure. In one or more embodiments, a biosensor is configured to sense a heart rate. In one or more embodiments, a biosensor is configured to sense a pulse oximetry reading. In one or more embodiments, a biosensor may be configured to sense a position of a subject in space.

In one or more embodiments, the devices, systems, and methods described herein are configured to transmit data regarding a subject to a remote healthcare provider or server. In an embodiment, the data transmitted comprises one or more biometric parameters of a subject. In an embodiment, the data transmitted comprises identifier data, such as, for example, a name, age, and gender of a subject. In an embodiment, the data transmitted comprises the position of a subject in space, such as, for example, whether the subject is upright or lying down.

In one or more embodiments, the devices, systems, and methods described herein are configured to provide a treatment to a subject. In an embodiment, a treatment is provided by a healthcare provider that is in contact with a subject needing treatment through a telemedical health encounter. In an embodiment, a healthcare provider recommends a therapy to a subject through a telemedical care encounter provided by the devices, systems, and methods described herein. In an embodiment, a healthcare provider provides a prescription through the telemedical care encounter provided by the devices, systems, and methods described herein. In an embodiment, the healthcare provider determines a therapy at least in part based on one or more biometric parameters transmitted by the devices and systems described herein and/or as part of a method described herein. In an embodiment, the devices, systems, and methods are configured to provide treatment to a subject by dispensing a medical item. In an embodiment, the medical item comprises a medication. In an embodiment, the devices and systems described herein are configured to contain a medication that may be dispensed by the devices and systems to the subject. In an embodiment, a healthcare provider remotely controls the devices and systems described herein so that the healthcare provider remotely causes the devices and systems described herein to dispense a medical item to a subject. In an embodiment, the subject causes the device and system to dispense the medical item. In an embodiment, the medical item contained in a device or system as described herein is provided to a subject ahead of the subject's need for the medical item.

In one or more embodiments, the devices, systems, and methods described herein are configured to operate in conjunction with electronically readable labels as well as electronically readable RFID tags and tokens.

All publications, patents, and patent applications mentioned in this specification are herein incorporated by reference to the same extent as if each subject publication, patent, or patent application was specifically indicated to be incorporated by reference.

### BRIEF DESCRIPTION OF THE DRAWINGS

The novel features of the subject matter are set forth with particularity in the appended claims. A better understanding of the features and advantages of the present subject matter will be obtained by reference to the following detailed description that sets forth illustrative embodiments, in which the principles of the subject matter are utilized, and the accompanying drawings of which:

FIG. 1 shows an embodiment of a device for providing a remote healthcare encounter comprising a wristlet.

FIG. 2 shows a system for providing a remote healthcare encounter.

FIG. 3 shows an embodiment of a device for providing a remote healthcare encounter comprising a tri-level docking smartwatch.

FIG. 4 shows an embodiment of a tri-level smartwatch and a system that includes the tri-level smartwatch.

FIG. 5 shows a device configured to provide a remote healthcare encounter comprising a pendant.

FIG. 6 shows an embodiment of the tri-level smart pendant.

FIG. 7 shows an embodiment of a device configured to provide a remote healthcare encounter comprising a bi-folding backplane dock for smartphones.

FIG. 8 shows embodiments of a bi-folding backplane dock for smartphones, laptop, and smartwatch as described herein.

FIG. 9 shows embodiments of a bi-folding backplane dock for smartphones, laptop, and smartwatch as described herein.

FIG. 10 shows embodiments of a laptop and smartwatch as described herein.

FIG. 11 shows embodiments of a computing device as described herein comprising a laptop.

FIG. 12 shows embodiments of a computing device as described herein comprising a laptop.

FIG. 13 shows embodiments of a laptop and smartwatch as described herein.

FIG. 14 shows embodiments of a bi-folding backplane dock for smartphones and smartwatch as described herein.

FIG. 15 shows embodiments of a laptop and smartwatch as described herein.

FIG. 16 shows an embodiment of a bi-folding backplane dock for smartphones and a container as described herein.

FIG. 17 shows an embodiment of a bi-folding backplane dock for smartphones as described herein.

FIG. 18 shows an embodiment of a bi-folding backplane dock for smartphones as described herein.

FIG. 19 shows embodiments of an adjustable bi-folding backplane dock for smartphones case as described herein.

FIG. 20 shows embodiments of an adjustable bi-folding backplane dock for smartphones case as described herein.

FIG. 21 shows embodiments of a bi-folding backplane dock for smartphones and a container as described herein.

FIG. 22 shows embodiments of a bi-folding backplane dock for smartphones and a container as described herein.

FIG. 23 shows embodiments of a bi-folding backplane dock for smartphones and a container as described herein.

FIG. 24 shows embodiments of a bi-folding backplane dock for smartphones and a container as described herein.

FIG. 25 shows embodiments of a bi-folding backplane dock for smartphones, docking units, and modules described herein.

FIG. 26 shows embodiments of a bi-folding backplane dock for smartphones, docking units, modules and a container as described herein.

FIG. 27 shows an embodiment of a bi-folding backplane dock for smartphones as described herein.

FIG. 28 shows an embodiment of a bi-folding backplane dock for smartphones as described herein.

FIG. 29 shows embodiments of a container, laptop and smartwatch as described herein.

FIG. 30 shows embodiments of a laptop and smartwatch as described herein.

FIG. 31 shows embodiments of a laptop and smartwatch as described herein.

FIG. 32 shows embodiments of a container, laptop and smartwatch as described herein.

### DETAILED DESCRIPTION OF THE INVENTION

Described herein are devices, systems, and methods for providing remote healthcare. Before explaining at least one embodiment of the inventive concepts disclosed herein in detail, it is to be understood that the inventive concepts are not limited in their application to the details of construction, experiments, exemplary data, and/or the arrangement of the components set forth in the following description, or illustrated in the drawings. The presently disclosed and claimed inventive concepts are capable of other embodiments or of being practiced or carried out in various ways. Also, it is to be understood that the phraseology and terminology employed herein is for purpose of description only and should not be regarded as limiting in any way.

In the following detailed description of embodiments of the described subject matter, numerous specific details are set forth in order to provide a more thorough understanding of the inventive concepts. However, it will be apparent to one of ordinary skill in the art that the inventive concepts within the disclosure may be practiced without these specific details. In other instances, well-known features have not been described in detail to avoid unnecessarily complicating the instant disclosure.

Further, unless expressly stated to the contrary, “or” refers to an inclusive or and not an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The term “subject” as used herein may refer to a human subject or any animal subject.

Finally, as used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Described herein are devices configured to provide remote healthcare. In an embodiment, a device for providing remote healthcare is configured to provide a telemedical care encounter. In an embodiment, a device for providing remote healthcare is configured to monitor a biometric parameter of a subject. In an embodiment, a device for providing remote healthcare is configured to transmit data regarding the subject to a remote healthcare provider. In an embodiment, a device configured to provided remote healthcare is further configured to electronically read a label, RFID chip, or magnetic strip associated with a subject and/or a medical item.

In an embodiment, a device for providing remote healthcare comprises a mobile computing device. In an embodiment, a mobile computing device comprises a smartphone or smartwatch.

In an embodiment, a device for providing remote healthcare comprises one or more of a display, one or more data ports configured to read one or more of a label or token. In an embodiment a label comprising information is affixed to a medical item. In an embodiment, the medical item is a medication container. In an embodiment, the medical item is a medical or surgical implement. In an embodiment the label comprises an adhesive sticker. The entire label may adhere or affix to a medical item. Alternatively, a portion of the label can adhere or affix to a medical item and a portion of the label can be not adhered or affixed to the medical item so that it freely hangs. In an embodiment, the label comprises a clear portion and an opaque portion. The opaque portion can comprise an information containing portion. An adhesive material can be associated with at least one surface of the clear portion. In an embodiment, the information containing portion of the label comprises a magnetic strip. In an embodiment, the information containing portion of the label comprises an RFID chip. In an embodiment, the information containing portion of the label comprises a contact based smart chip. In an embodiment, the information containing portion of the label comprises either a barcode or a two-dimensional barcode. In an embodiment, the information containing portion comprises one or more of a magnetic strip, an RFID chip, a contact based smart chip, a barcode, or a two-dimensional barcode. In an embodiment, the information containing portion comprises a means for transmitting an RF or Bluetooth signal. In an embodiment, the information containing portion of the label comprises information that comprises at least one of subject information data, medication information data, information relating to an implement, and payment data. Payment data may comprise information that will provide for a payment transfer from the account of a subject to the account of a payee when the subject's payment information is accessed. Payment information can be for example credit card information. In an embodiment, a data reader associated with a device for providing a remote healthcare encounter reads information from the information containing portion of the label through a magnetic strip reader. In an embodiment, the device for providing a remote healthcare encounter reads information from the information containing portion of the label through an RFID chip reader. In an embodiment, the device for providing a remote healthcare encounter reads information from the information containing portion of the label through a contact based smart chip reader. In an embodiment, the device for providing a remote healthcare encounter for providing a remote healthcare encounter reads information from the information containing portion of the label through a barcode reader. In an embodiment, the device for providing a remote healthcare encounter for providing a remote healthcare encounter, reads information from the information containing portion of the label through a two-dimensional barcode reader. In an embodiment, the electronic for providing a remote healthcare encounter device comprises one or more of a magnetic strip reader, an RFID chip reader, a contact based smart chip reader, a barcode reader, or a two-dimensional barcode reader. In an embodiment, the reader associated with a device for providing a remote healthcare encounter comprises a means for receiving an RF or Bluetooth signal. In an embodiment, a label can be linked to a device for providing a remote healthcare encounter by engaging the information containing portion of the label with the reader means of the device for providing a remote healthcare encounter.

In an embodiment, the device for providing a remote healthcare encounter comprises a screen that displays information to a user. The information displayed can be displayed as a menu. The menu can be an interactive menu. The menu can display information organized in accordance with a typical menu structure. The menu can display information comprising insurance or guarantee information for certain events, medication or prescription renewal information, medication refill information, appointment information, payment information, alternative medicine information, and a means for transmitting a question to a healthcare provider. The insurance or guarantee information can comprise an offer to purchase insurance or guarantees for a short term event. A short term event can be an action related to a medical item associated with the labeling system, such as the taking of a medication to which the label of the labeling system is affixed or adhered. The payment information can comprise the price of the medical item to which the label of the labeling system is affixed. The menu can display an alternative medication for the subject to take instead of the one to which the label is affixed. The alternative medication can be determined using an economic risk assessment, wherein the economic risk comprises the risk that a subject would overpay for a medical item with a less expensive alternative, such as a generic medication.

In an embodiment, a healthcare provider at a remote location is able to communicate with a subject through the device for providing a remote healthcare encounter. In an embodiment, the subject and the healthcare provider are able to communicate with each other. The communications can be in real time in one or more of text, audio, or visual formats.

In an embodiment, the medical item associated with a label is delivered to the subject before the medical item is needed based on an anticipated future need for the medication. Determination of an anticipated need can be based on the statistical level of likelihood that the items will be needed in the short term future based on various subject-related and environmental factors.

In further various embodiments, anticipated need is based on statistical level of likelihood that the items will be needed within, by way of non-limiting examples, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days, including increments therein. In further various embodiments, anticipated need is based on statistical level of likelihood that the items will be needed within, by way of non-limiting examples, 1, 2, 3, 4, 5, 6, 7, 8, 9, 10, 11, 12, 13, 14, 15, 16, 17, 18, 19, 20, 21, 22, 23, 24 or more months, including increments therein. In an embodiment, ahead in time delivery enables the systems, devices, and methods described herein to provide real in time therapy.

In an embodiment, the subject links the medical label with the device for providing a remote healthcare encounter at the time that the use of the medical item-which was provided before the subject needed it-becomes indicated. For example, a subject with no heart symptoms who is told to take a heart medication if the subject has onset of specified symptoms, such as chest pain or dizziness, would link the label of the labeling system with the device for providing a remote healthcare encounter on or around the time of taking the medication.

In an alternative embodiment a token system comprises a token that comprises an information containing portion and a device for providing a remote healthcare encounter that links with the token.

In an embodiment the token is directly connected to a medical item. In an embodiment the token is not connected to a medical item. The token can be either adhered or affixed to a medical item, it can be entirely not connected to a medical item, or it can be detachably connected to a medical item. A token can be associated with a single particular medical item to which it is not directly connected. For example, a token associated with a subject's particular medication can be connected to the subject's key chain.

In an embodiment the token comprises of a fob. In an embodiment the token comprises of a magnetic strip. In an embodiment the information containing portion of the token comprises an RFID chip. In an embodiment the information containing portion of the token comprises a contact based smart chip. In an embodiment the information containing portion of the token comprises either a barcode or a two-dimensional barcode. In an embodiment the information containing portion comprises one or more of a magnetic strip, an RFID chip, a contact based smart chip, a barcode, or a two dimensional barcode.

In an embodiment the information containing portion comprises a means for transmitting an RF or Bluetooth signal.

In an embodiment the information containing portion of the token comprises information that comprises at least one of subject information data, medication information data, information relating to an implement, and payment data. Payment data can comprise information that will provide for a payment transfer from the account of a subject to the account of a payee when the subject's payment information is accessed. Payment information can be for example credit card information.

In an embodiment the device for providing a remote healthcare encounter comprises a processor, a memory, a data reader, a screen, and software. In an embodiment the device for providing a remote healthcare encounter further comprises an interactive user interface. In an embodiment the device for providing a remote healthcare encounter further links to other device for providing a remote healthcare encounters or the internet.

In an embodiment the data reader reads information from the information containing portion of the token through a magnetic strip reader. In an embodiment the device for providing a remote healthcare encounter reads information from the information containing portion of the token through an RFID chip reader. In an embodiment the device for providing a remote healthcare encounter reads information from the information containing portion of the token through a contact based smart chip reader. In an embodiment the device for providing a remote healthcare encounter reads information from the information containing portion of the token through a barcode reader. In an embodiment the device for providing a remote healthcare encounter reads information from the information containing portion of the token through a two dimensional barcode reader. In an embodiment the device for providing a remote healthcare encounter comprises one or more of a magnetic strip reader, an RFID chip reader, a contact based smart chip reader, a barcode reader, or a two-dimensional barcode reader.

In an embodiment the reader comprises a means for receiving an RF or Bluetooth signal.

A token can be linked to a device for providing a remote healthcare encounter by engaging the information containing portion of the token with the reader means of the device for providing a remote healthcare encounter.

In an embodiment the device for providing a remote healthcare encounter comprises a screen that displays information to a user. The information displayed can be displayed as a menu. The menu can be an interactive menu. The menu can display information organized in accordance with a typical menu structure. The menu can display information comprising insurance or guarantee information for certain events, medication or prescription renewal information, medication refill information, appointment information, payment information, alternative medicine information, and a means for transmitting a question to a healthcare provider. The insurance or guarantee information can comprise an offer to purchase insurance or guarantees for a short term event. A short term event can be an action related to a medical item associated with the token system, such as the taking of a medication to which the label of the token system is affixed or adhered. The payment information can comprise the price of the medical item to which the token of the token system is affixed. The menu can display an alternative medication for the subject to take instead of the one to which the token is affixed. The alternative medication can be determined using an economic risk assessment, wherein the economic risk comprises the risk that a subject would overpay for a medical item with a less expensive alternative, such as a generic medication.

In an embodiment a healthcare provider at a remote location is able to communicate with a subject through the device for providing a remote healthcare encounter. In an embodiment the subject and the healthcare provider are able to communicate with each other. The communications can be in real time in one or more of text, audio, or visual formats.

In an embodiment, the medical item associated with the token system is delivered to the subject before the medical item is needed based on an anticipated future need for the medication. Determination of an anticipated need can be based on the statistical level of likelihood that the items will be needed in the short-term future based on various subject-related and environmental factors.

In further various embodiments, anticipated need is based on statistical level of likelihood that the items will be needed within, by way of non-limiting examples, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days, including increments therein. In further various embodiments, anticipated need is based on statistical level of likelihood that the items will be needed within, by way of non-limiting examples, 1, 2, 3, 4, 5, 6, 7, 8, 9, 10, 11, 12, 13, 14, 15, 16, 17, 18, 19, 20, 21, 22, 23, 24 or more months, including increments therein. In an embodiment, ahead in time delivery enables the systems, devices, and methods described herein to provide real in time therapy.

In an embodiment the subject links the medical token with the device for providing a remote healthcare encounter at the time that the use of the medical item-which was provided before the subject needed it-becomes indicated. For example, a subject with no heart symptoms who is told to take a heart medication if the subject has onset of specified symptoms, such as chest pain or dizziness, would link the token of the token system with the device for providing a remote healthcare encounter on or around the time of taking the medication.

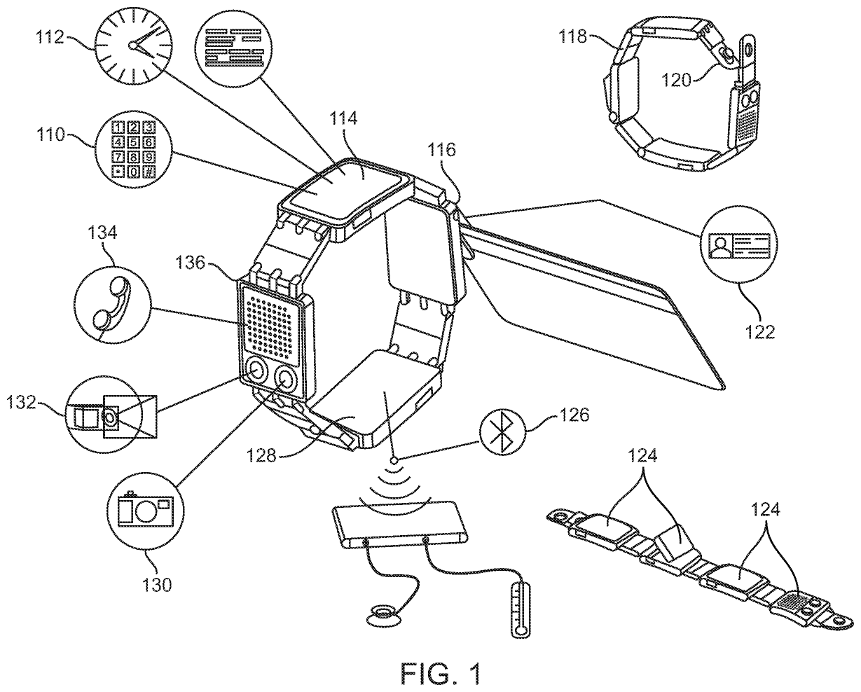

FIG. 1 shows an embodiment of a device for providing a remote healthcare encounter comprising a wristlet. In an embodiment, a wristlet comprises a smartwatch such as, for example, the Apple Watch. In an embodiment, a wristlet comprises of multiple units. In an embodiment, the multiple units may comprise a main interface screen 114, a communications unit 136, a connectivity screen or unit 126, and a data port 116. In an embodiment, a main interface screen 114 may comprise a variable touch screen and/or keypad. In an embodiment, the main interface screen 114 is configured to, for example, provide one or more of a clock, phone touchpad, or readable text and/or images and icons. In an embodiment, a communications unit 136 comprises one or more of a phone speaker and/or receiver 134, a projector 132, and a camera 130, wherein the camera 130 may be further configured to provide real-time video recording. In an embodiment, a connectivity screen or unit 126 is configured to transmit and/or receive data. In an embodiment, a connectivity screen or unit 126 is configured to transmit data relating to a subject to a computing device that may be further configured to comprise biometric sensors. In an embodiment, a data port 116 comprises a reader as described herein that may be, for example, configured to read a magnetic strip from a subject's identification or payment card. In an embodiment, a wristlet 124 may be custom configured to include one more of the units described in accordance with the needs of the subject.

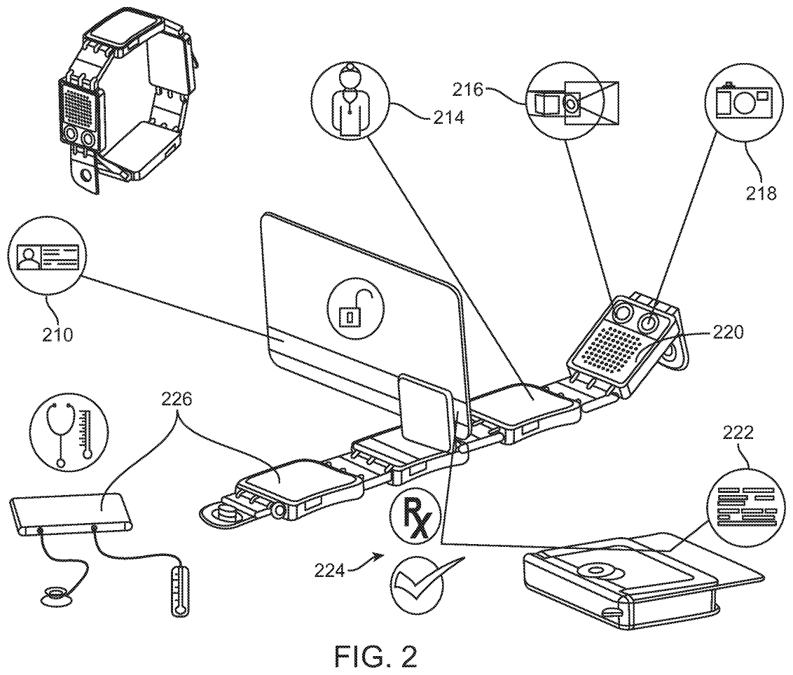

FIG. 2 shows a system for providing a remote healthcare encounter. In an embodiment, a system for providing a healthcare encounter comprises a device for providing a healthcare encounter such as the wristlet as shown in FIG. 1. In an embodiment, a system of providing a healthcare encounter may comprise a device for providing a healthcare encounter comprising, for example, a smartwatch or smartphone instead of the wristlet as shown in FIG. 1. In an embodiment, the device for providing a healthcare encounter (shown here as a wristlet) comprises one or more embedded diagnostic tools comprising biometric sensors. In an embodiment, a subject interacts either a data containing label or token with a data port/data reader as described herein. In an embodiment, data read off of a label or token (or magnetic stripe 210 as shown) is received by the device for providing a remote healthcare encounter and transmitted to, for example, a container 222 that contains a medical item. In an embodiment, the data transmitted from the device for providing a remote healthcare encounter causes the container 222 to dispense a medical item contained therein. In an embodiment, the container 222 further comprises a display screen configured to display, for example, data relating to the subject and/or data relating to the medical item contained therein such as, for example, drug label information for a dispensed medication or instructions for a dispensed non-medication medical item.

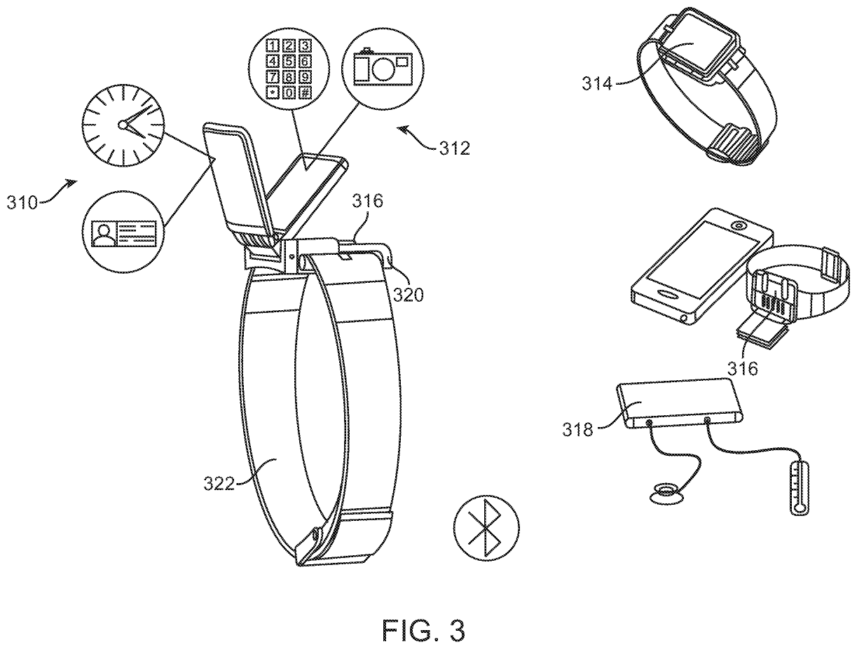

FIG. 3 shows an embodiment of a device for providing a remote healthcare encounter comprising a tri-level docking smartwatch. In an embodiment, the tri-level docking smartwatch comprises three tiers: tier I 310, tier-2 312, and tier-3 316. In an embodiment, the three tiers of a tri-level docking smartwatch may be stacked on top of one another with the tier-I comprising the topmost level, tier-2 comprising the middle layer, and tier-3 comprising the lowest layer. In an embodiment, a hinge allows tiers 1 and 2 to flip up and rotate away from tier-3 and from each other by pivoting around the hinge. It should be understood that the embodiments described herein are not limited to 3 such tiers but may be either 2 such tiers or 4 or more such tiers. It should be also understood that in alternative embodiments a hinge mechanism may provide rotational motion of the individual tiers so that they rotate or fan out away from each other. In an embodiment, tier-I 310 comprises a double sided screen that, for example, displays a clock and/or subject identifying information. In an embodiment, tier-2 312 comprises an interface with, for example, a variable touch screen, keypad, sensors, print reader, and/or a camera. In an embodiment, tier-3 316 comprises dock tools and devices that may be, for example, configured to read, scan, receive and transmit data. In an embodiment, a tri-level docking smartwatch comprises a data reader 320 configured to acquire data through one or more of a magnetic strip reader, an RF-ID reader, or a barcode reader. In an embodiment, a tri-level smartwatch is configured to wirelessly transmit a signal through, for example, Bluetooth.

In an embodiment a tri-level smartwatch as shown in FIG. 3 is part of a system that further comprises one or more of a mobile computing device configured to receive and transmit data with the tri-level smartwatch, a container containing a medical item as, for example, described in FIG. 2, and/or biometric sensors configured to receive and transmit data with the tri-level smartwatch.

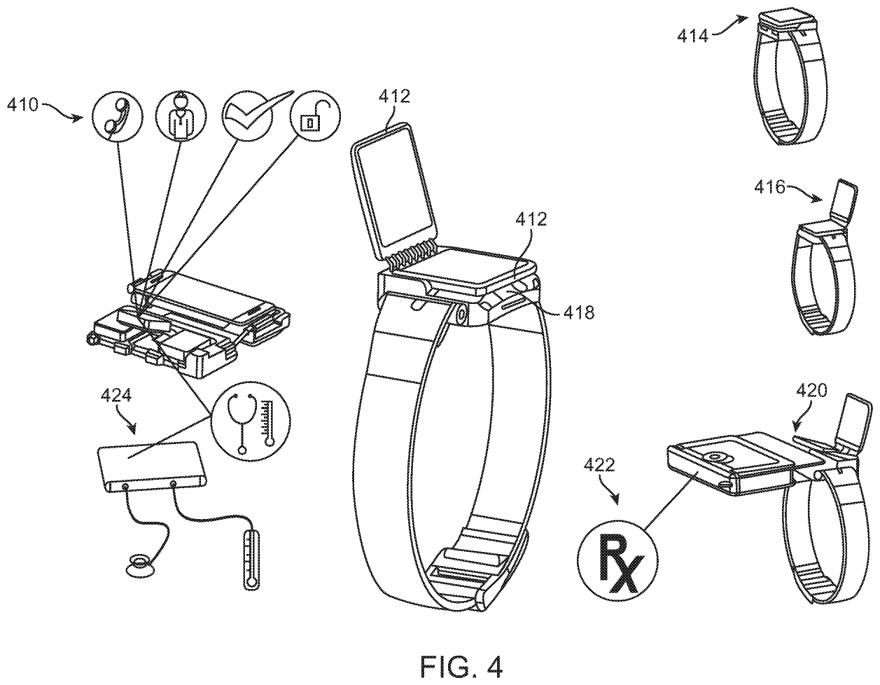

FIG. 4 shows an embodiment of a tri-level smartwatch and a system that includes the tri-level smartwatch. In an embodiment, position 414 comprises all three tiers of the tri-level smartwatch stacked one on top of the other, with one side of the tier-I comprising a visible display screen. In an embodiment, a position 416 comprises tier-I flipped open with tier-2 stacked on top of tier-3. In an embodiment, position 416 of the tri-level smartwatch is configured to provide a telemedical care encounter, wherein, for example, a camera on tier-I or records and transmits an image of a subject and a display on one side of tier-I displays a remote healthcare provider. In an embodiment, position 420 comprises both tier-I and tier-2 flipped away from tier-3, thus fully exposing the docking element on tier-3. In an embodiment, the docking feature on tier-3 is configured to operably couple with a container 422 as described herein to, for example, cause said container 422 to dispense a medical item. In an embodiment, the interaction between the docking element on tier-3 of the tri-level smartwatch and the container 422 is controlled at least in part remotely by the remote healthcare provider. That is, for example, in an embodiment, a remote healthcare provider transmits data to the tier-3 dock that enables the tier-3 dock to properly couple with container 422 such that container 422 dispenses a medical item contained therein. In an embodiment, a tri-level smartwatch may be configured to communicate with another types of dispensers and docks containing various modules.

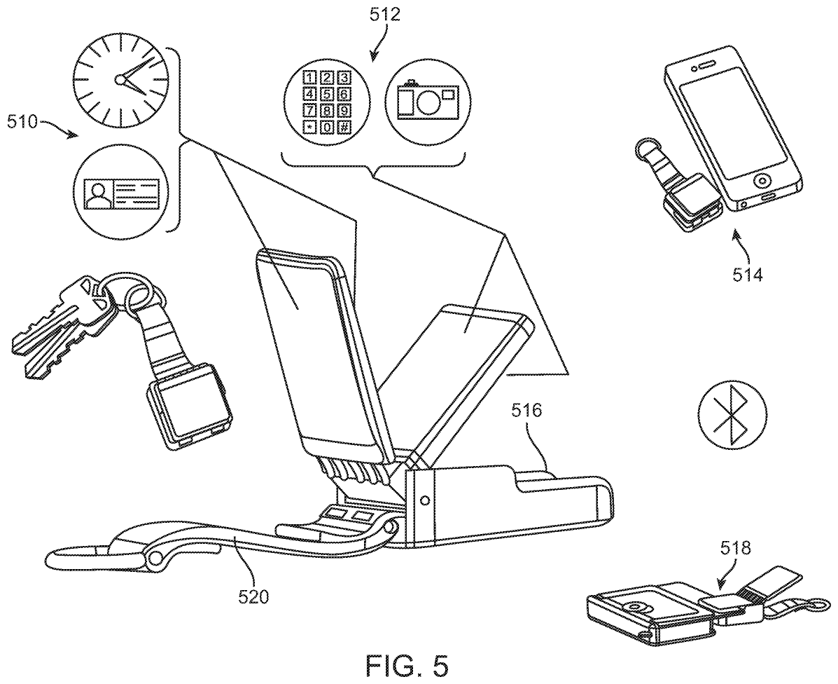

FIG. 5 shows a device configured to provide a remote healthcare encounter comprising a pendant. In an embodiment, a tri-level pendant is similar to a tri-level smartwatch as shown in FIGS. 3-4. In an embodiment, a tri-level pendant comprises three tiers: Tier-I 510 configured to open and close and may provide, for example, a double display screen, tier-2 configured to open and close and may provide, for example, a variable touch pad, sensors, and a print reader, and tier-3 which may be, for example, configured to provide dock tools. It should be understood that the embodiments described herein are not limited to 3 such tiers but may be either 2 such tiers or 4 or more such tiers. It should be also understood that in alternative embodiments a hinge mechanism may provide rotational motion of the individual tiers so that they rotate or fan out away from each other.

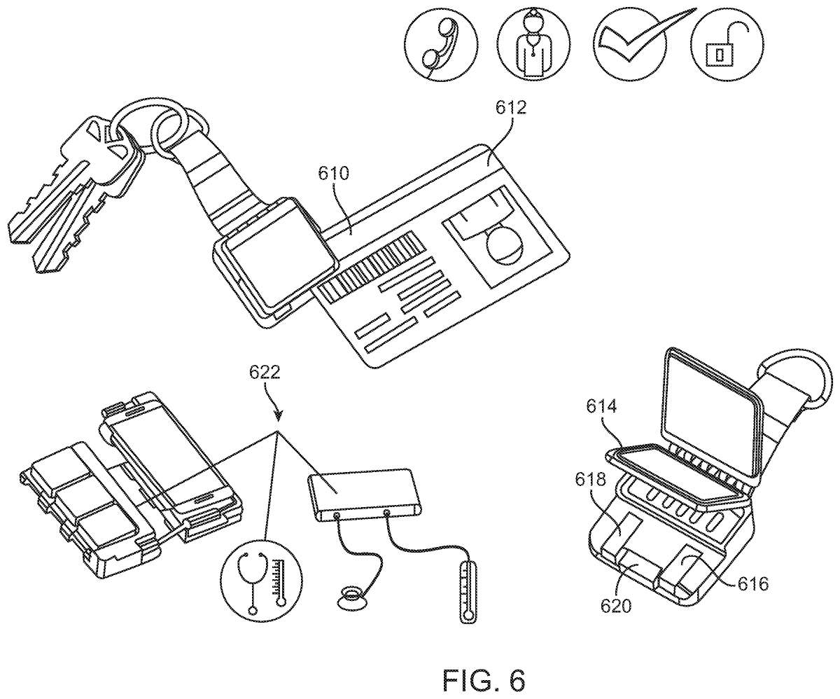

FIG. 6 shows an embodiment of the tri-level smart pendant. In an embodiment, a tier-3 comprises a dock that is configured to read a magnetic strip 612. In an embodiment, a docking unit may, for example, comprise one or more of ports for special equipment 614, a magnetic strip reader 616, an RF-ID reader, a bar code reader 618, and a bio reader 620.

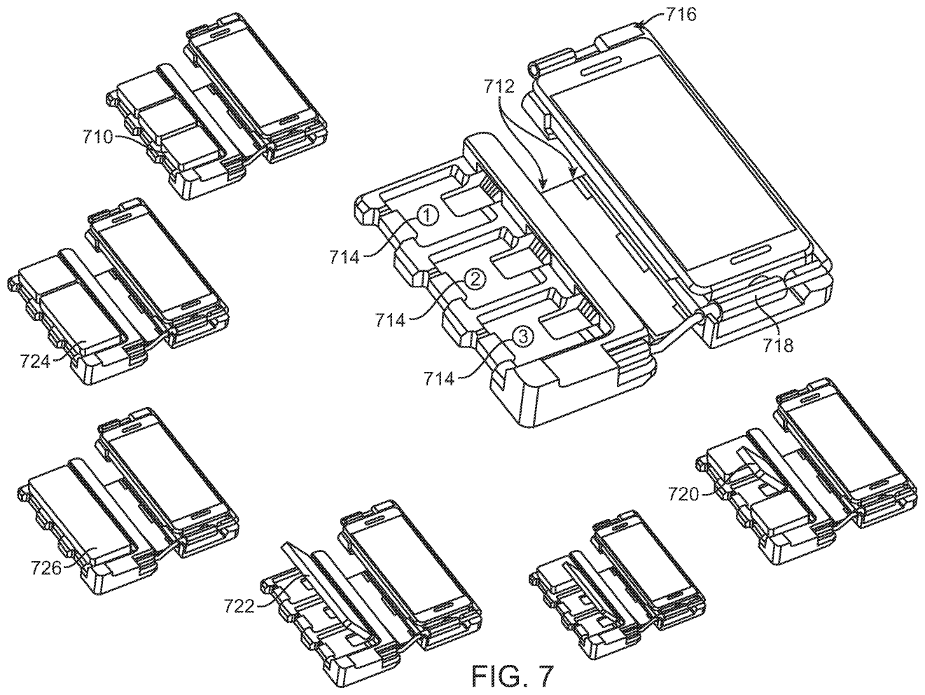

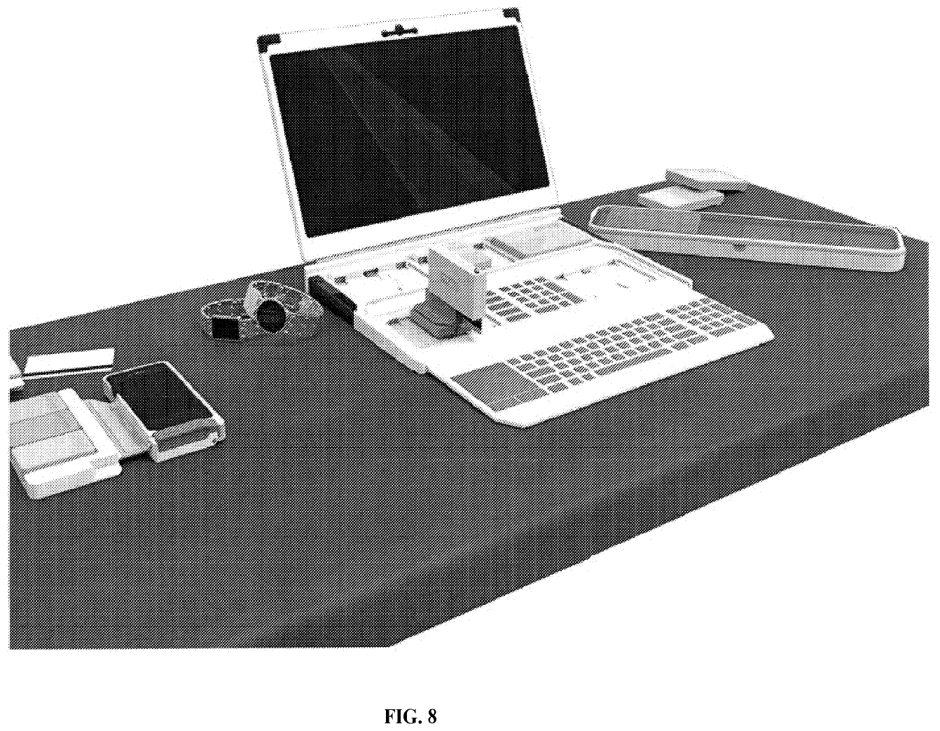

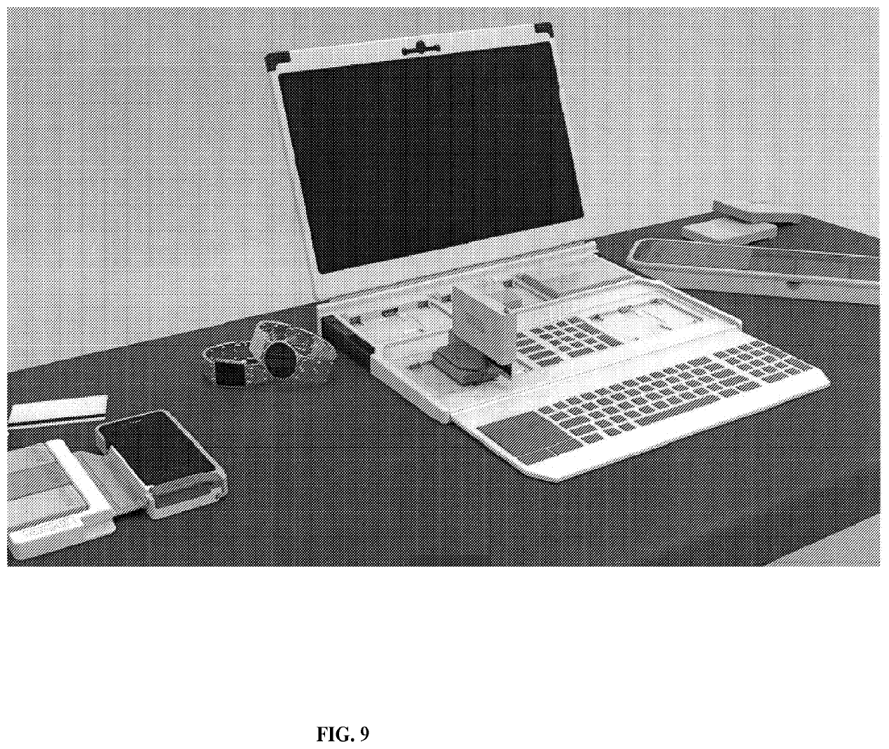

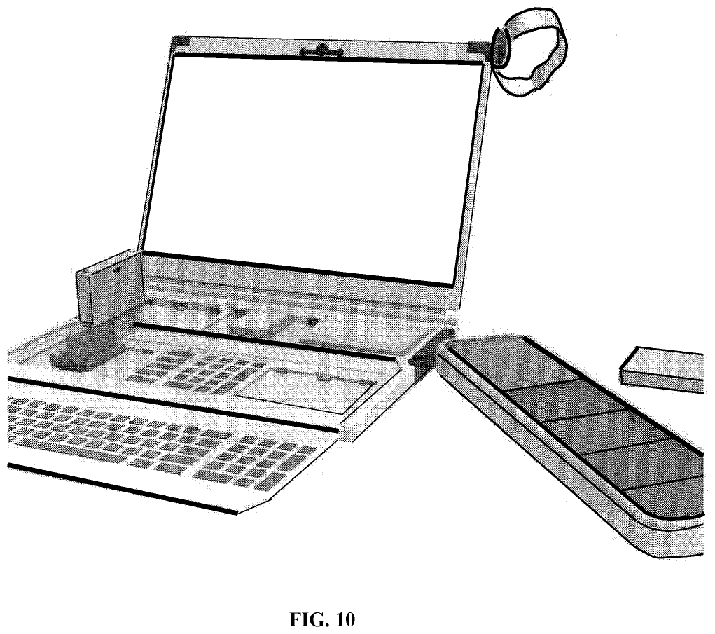

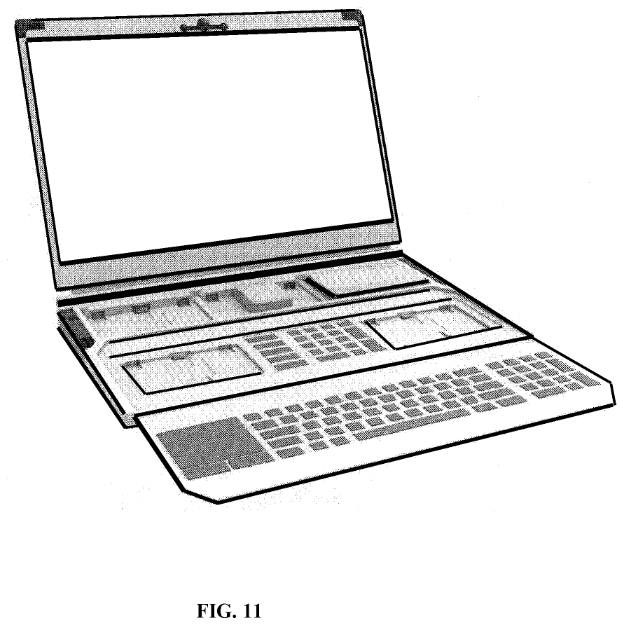

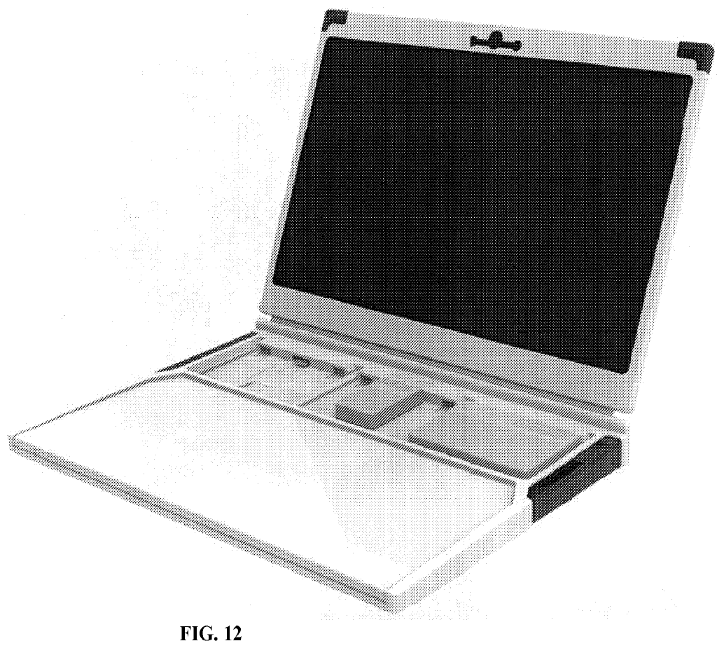

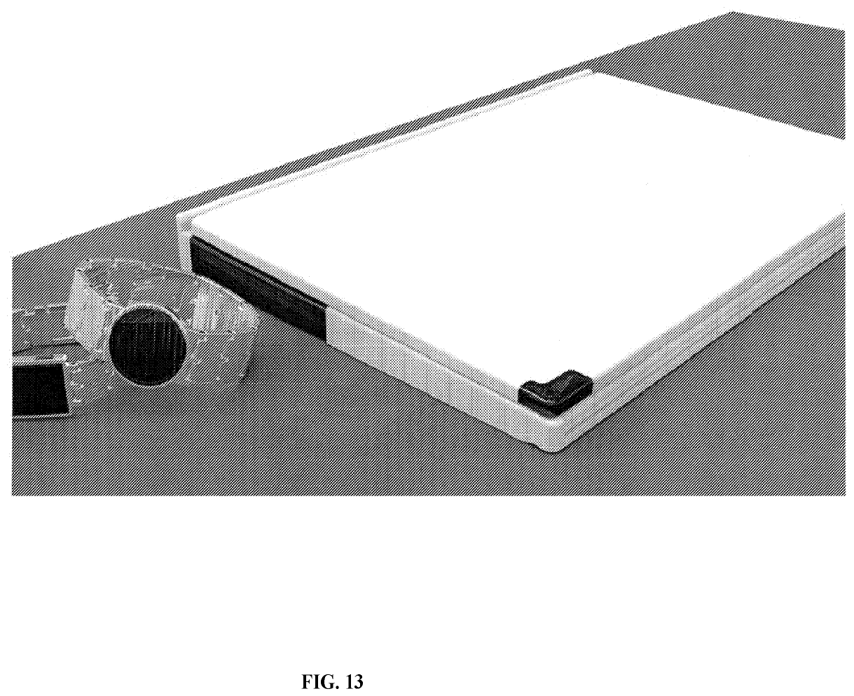

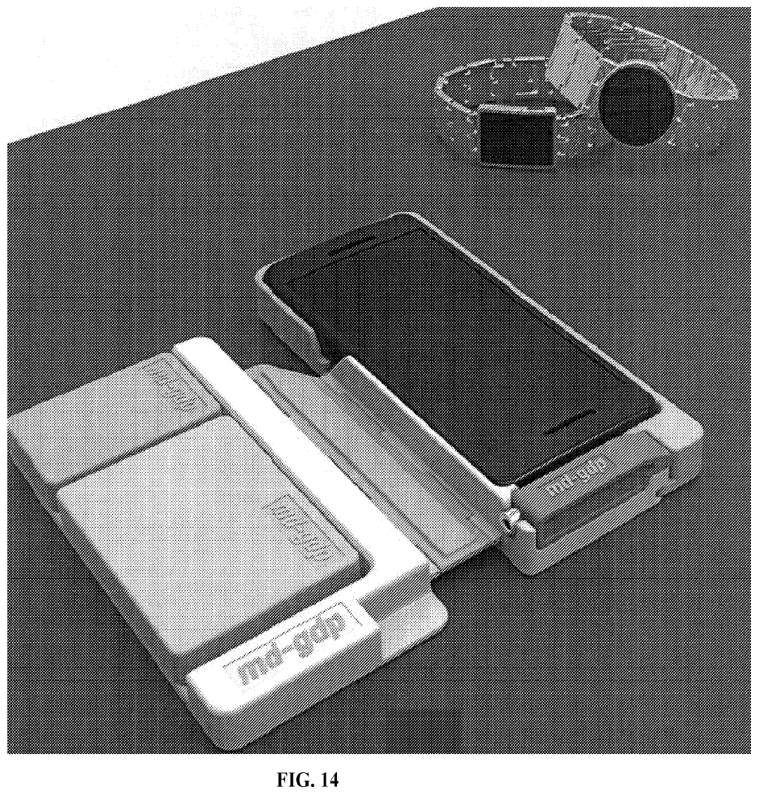

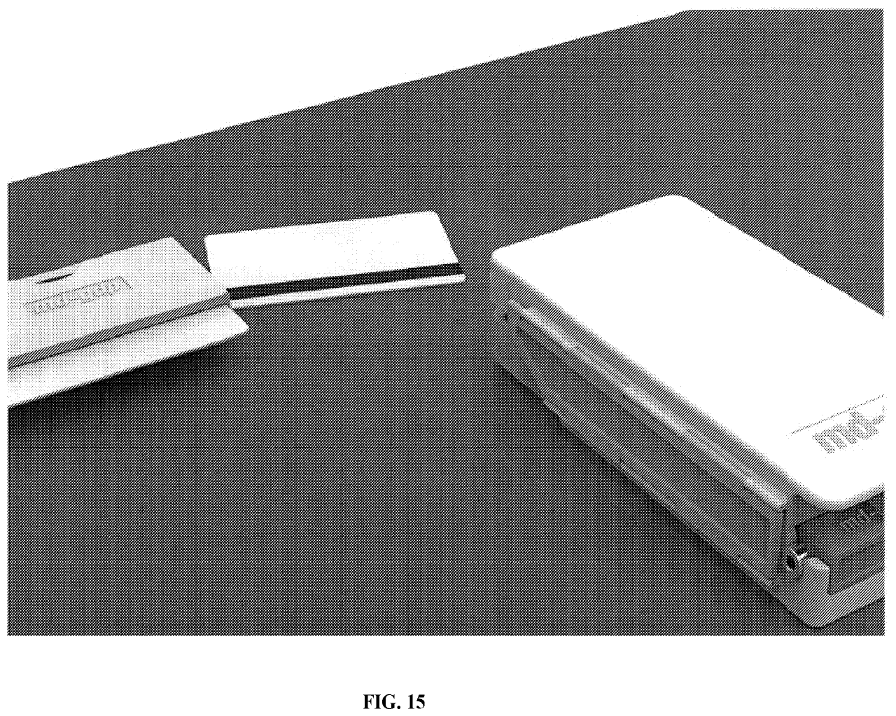

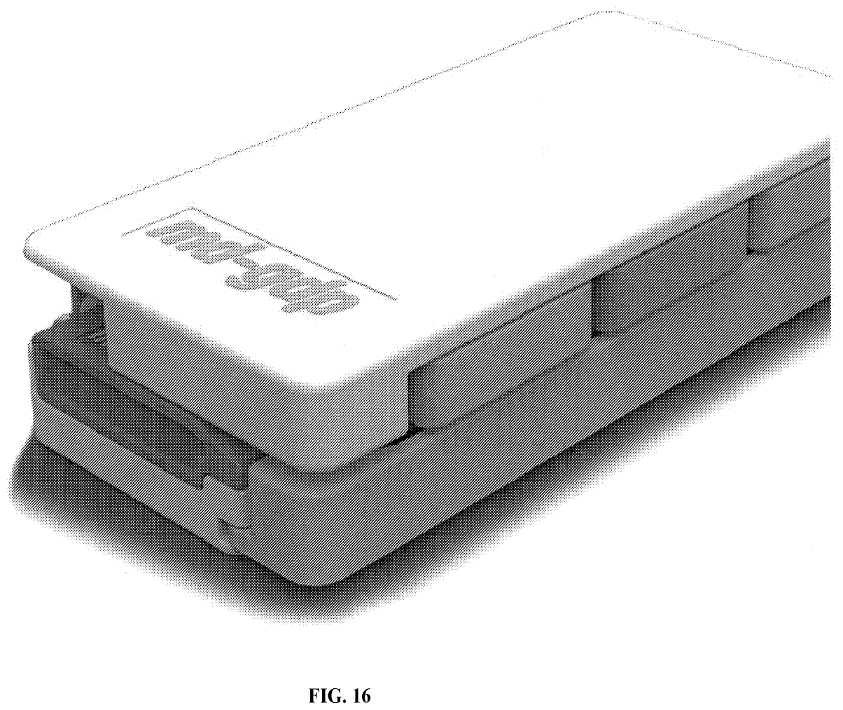

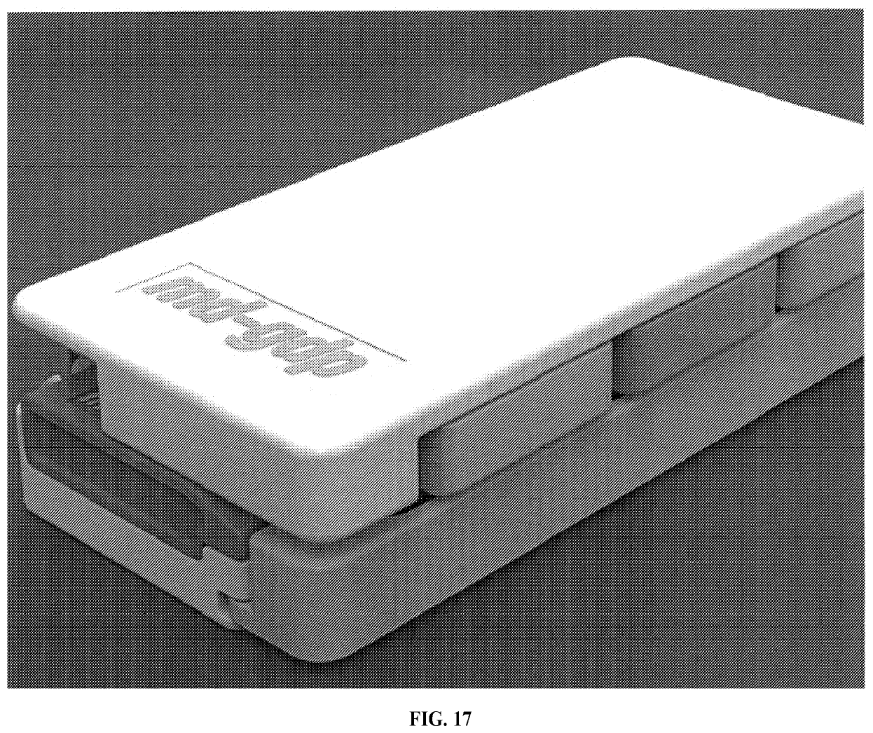

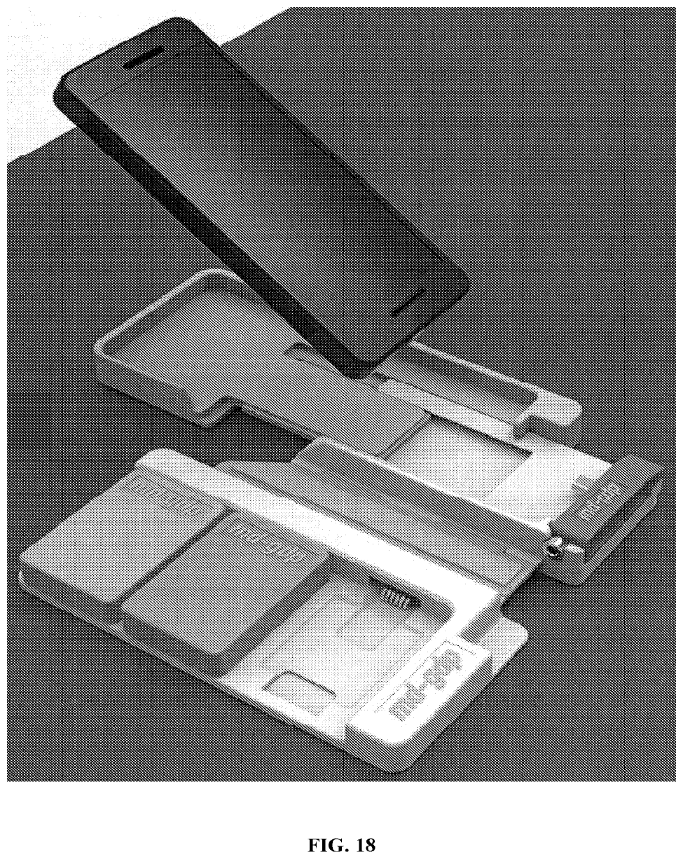

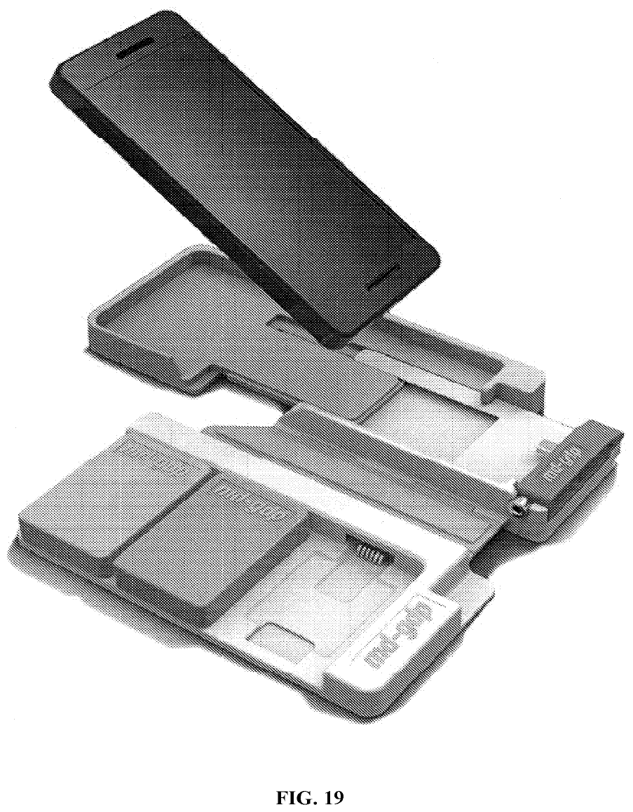

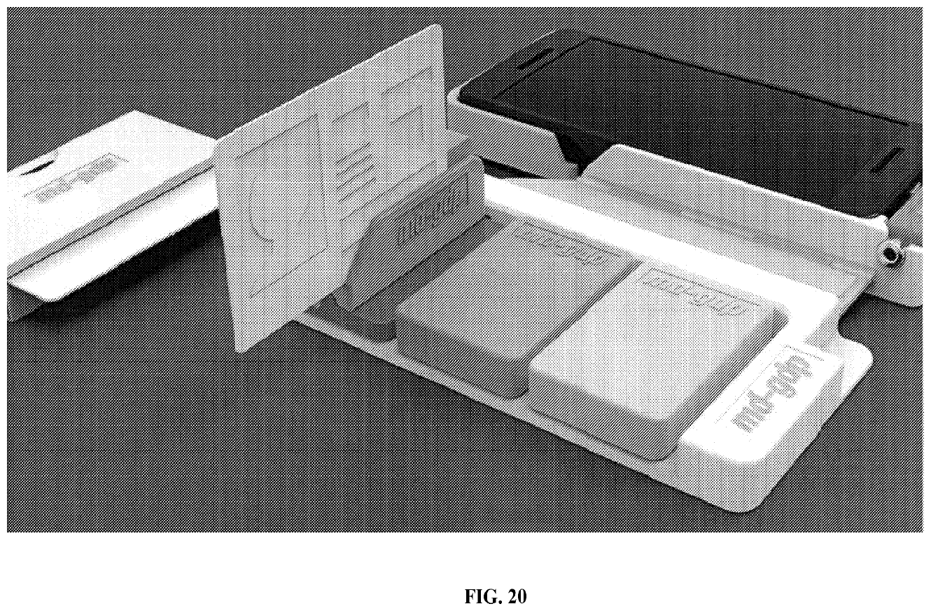

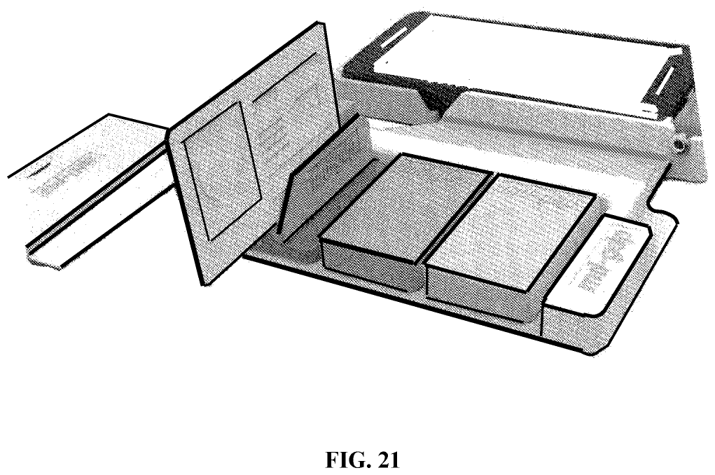

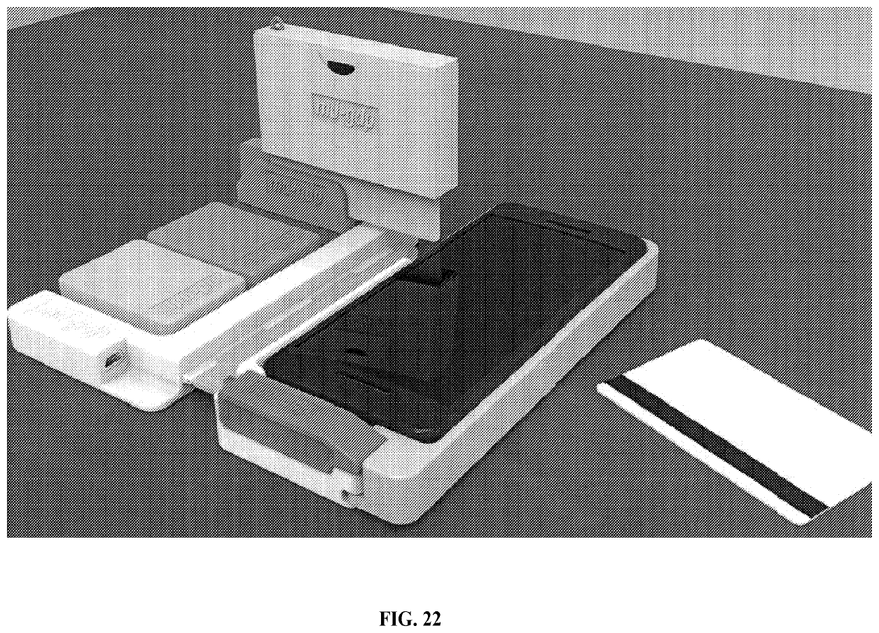

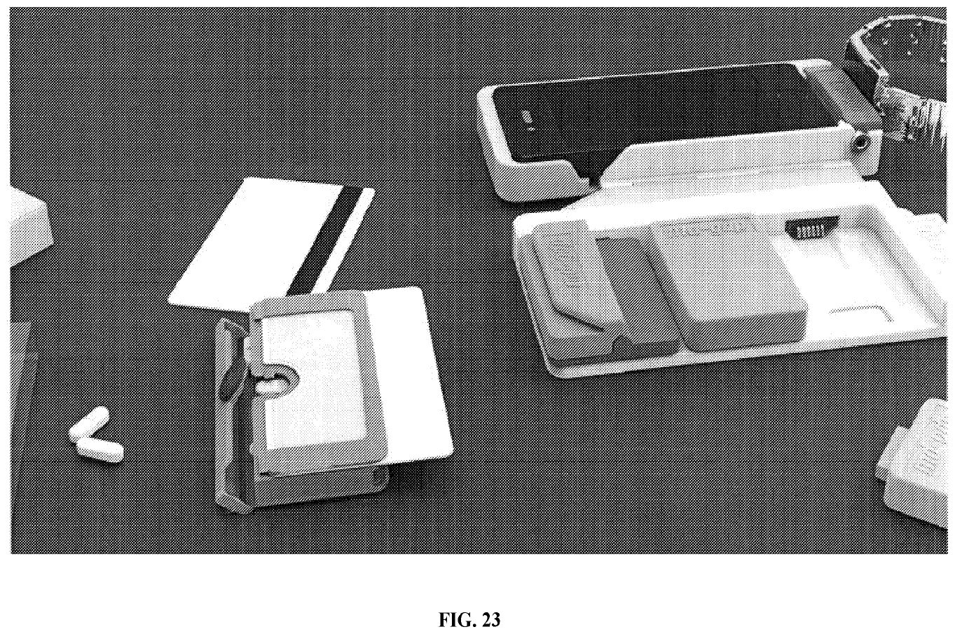

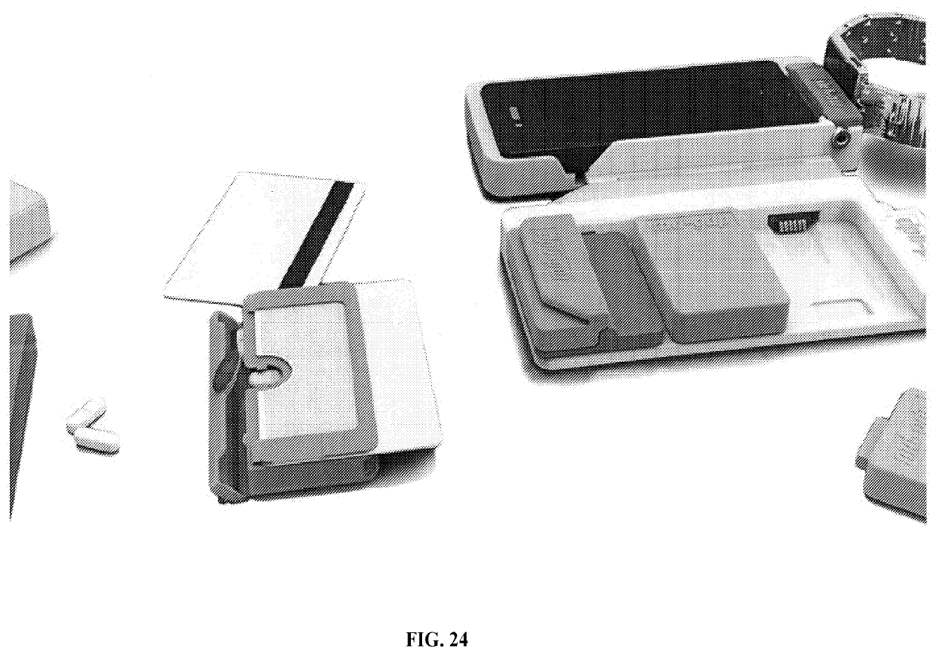

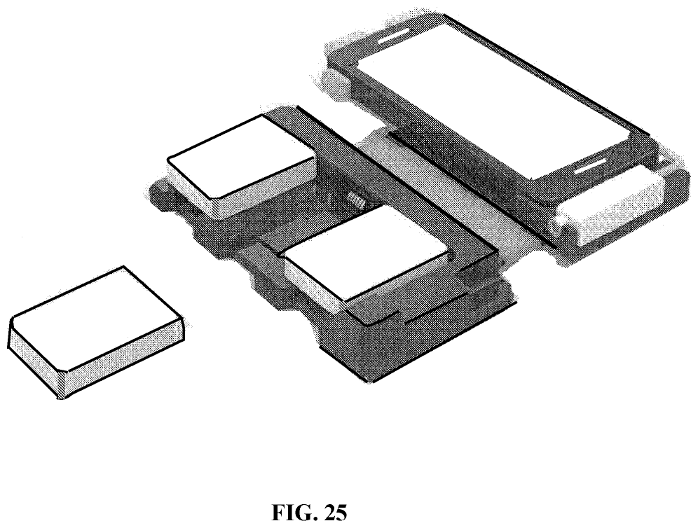

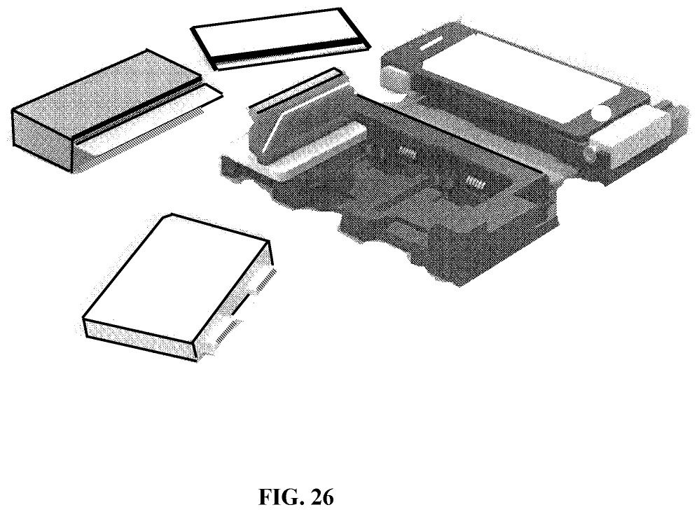

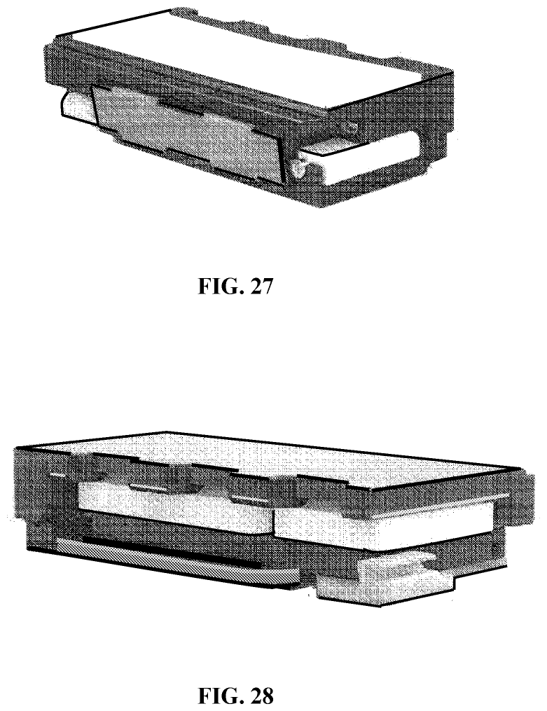

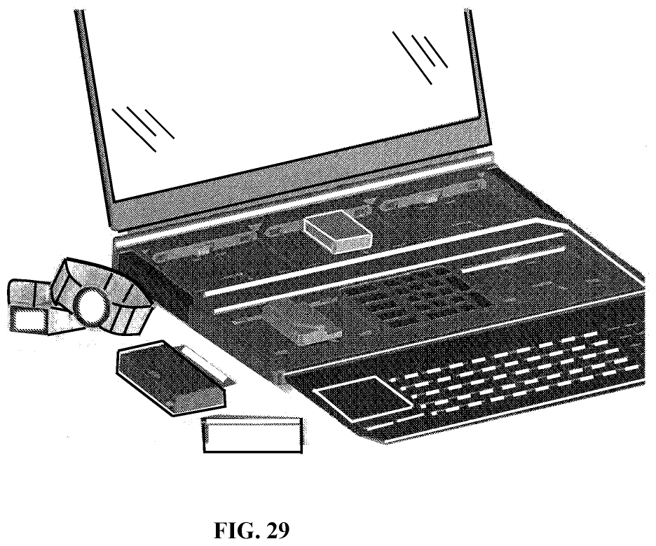

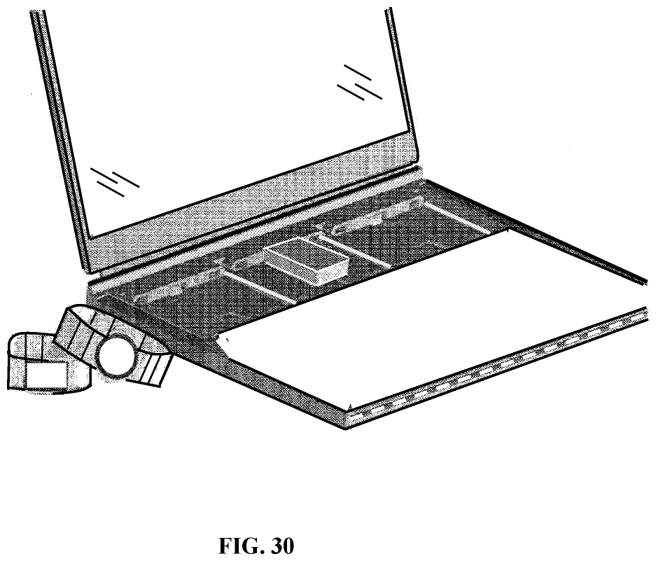

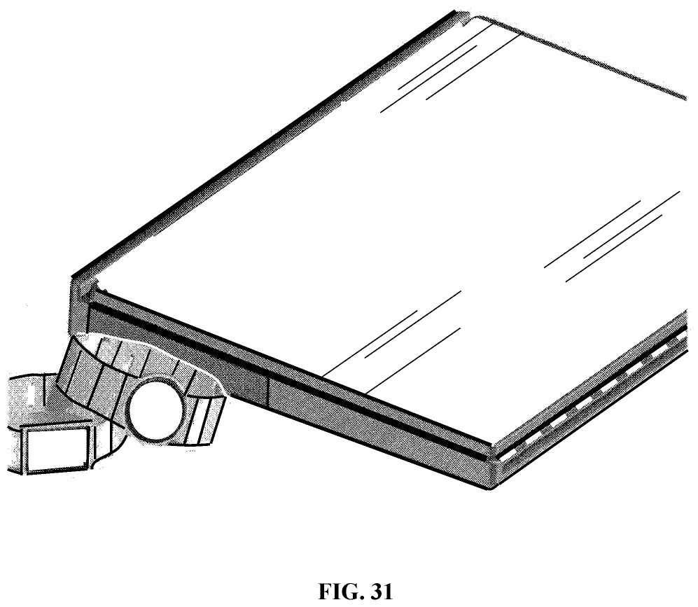

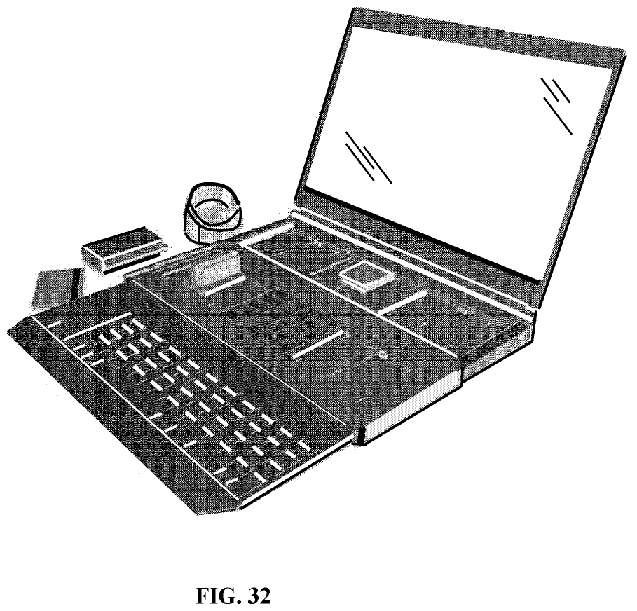

FIG. 7 shows an embodiment of a device configured to provide a remote healthcare encounter comprising a bi-folding backplane dock for smartphones. In an embodiment, a bi-folding backplane dock for smartphones comprises a folding case that couples with and integrates with a smartphone or another mobile computing device. In an embodiment, a bi-folding backplane dock for smartphones comprises double hinges 712. In an embodiment, a bi-folding backplane dock for smartphones comprises one or more universal docking slots that are configured to contain one or more modules, wherein the one or more modules may, for example, contain medical items. In an embodiment, a bi-folding backplane dock for smartphones is adjustable so that it may be coupled with smartphones and mobile computing devices of different sizes. In an embodiment, a data adapter 718 that is configured to plug into a port on a smartphone or other mobile computing device is interchangeable so that a data adapter can be matched to the type of smartphone or other mobile computing device coupled with the bi-folding backplane dock for smartphones. In an embodiment, a module comprises a device that couples with one or more of the universal docking slots 714. In an embodiment, the device that couples with the one or more universal docking slots 714 comprises a biometric sensor.

As an example in a preferred embodiment in order to count the medications a subject is reporting as taken a label on the medication, or medical supply or a non-medically related purchased item is used to create a more accurate electronic audit trail, an embodiment of a label attachable physically or by software or by near field or far field electronic connectivity container a near field exercisable token configured to store information comprising subject identification data and information data, the token further configured to be functionally linked to an electronic device comprising a memory, a processor, a data reader configured to read or receive information from the token, and a screen having an accelerometer to record a user performed tap of a token onto a screen to impart a force onto an accelerometer of a token onto said screen as an assignable counted event related to an action or use of the function of an item to which a token is attached or electronically related with software to a database and software configured to display an interactive menu comprising information read from or transmitted from the token, wherein the electronic device is configured to link to a second electronic device or the internet. Specific features software and firmware programmed existing printers that are reconfigured as well as unique printers newly manufactured and that do not use legacy printers as primary to create the labels with the tokens embedded and readers of the tokens as within cellphones, in separate devices like for example a watch or table top reader that is linked with near field and internet or 4G like phone connectivity to the parties involved and their databases.

While preferred embodiments of the present subject matter have been shown and described herein, it will be obvious to those skilled in the art that such embodiments are provided by way of example only.

Numerous variations, changes, and substitutions will now occur to those skilled in the art without departing from the subject matter. It should be understood that various alternatives to the embodiments of the subject matter described herein may be employed in practicing the subject matter. It is intended that the following claims define the scope of the subject matter and that methods and structures within the scope of these claims and their equivalents be covered thereby.

We claim:

1. A system comprising:
a medical item anticipated to be needed by a subject at a future point in time, the medical item having a label or token directly or indirectly attached thereto and detachable therefrom, wherein the medical item is selected based on a statistical level of likelihood that the medical item will be needed by the subject at the future point in time using one or more of a present biometric data about the subject or an environmental factor, wherein the label or token is electronically indexed to an electronic record, and wherein the label or token includes an information portion having an information identifying the medical item, an information identifying the contents of the medical item, an information about the subject, an information about a medication in the medical item, or a combination thereof; and
an electronic device for obtaining the information or combination of information from the label or token, the electronic device comprising media-stored and processor-executable software adapted for communicating the information or combination of information to the electronic device when the label or the token and the electronic device are proximate to each other, identifying when a physical tap of the token is made on a portion of the electronic device, and causing the electronic storing of a cumulative count of the physical tap or a use of the medical item.

2. The system of claim 1, wherein the token comprises one of a medication label, a fob, a magnetic strip, a barcode, or a smart chip.

3. The system of claim 1, wherein the information or combination of information further comprises a security code and a payment data.

4. The system of claim 1, wherein the software is further adapted to displaying using the electronic device an interactive menu including displaying at least some of the information or combination of information.

5. The system of claim 4, wherein the software is further adapted to displaying using the electronic device an interactive menu including displaying the result of the selection of the medical item.

6. The system of claim 1, wherein the token includes an RFID chip or a contact-based smart chip adhered or affixed to the medical item or detachably connected to the medical item.

7. The system of claim 1, wherein the information portion of the label is a magnetic strip and the communications portion of the electronic device is a magnetic strip reader, or the information portion of the label is a barcode and the communications portion of the electronic device is a barcode reader.

8. The system of claim 1, wherein the software is further adapted to communicating an information with a second electronic device or electronically connecting to a network.

9. The system of claim 1, further comprising:
a device connected to the electronic device, the device comprising one or more docking slots for receiving respective one or more modules selected from a module for storing the medical item and a module for containing a biometric sensor for outputting the biometric data.

10. The system of claim 9, wherein the software is further adapted to sending the biometric data about the subject obtained from the biometric sensor to a remote healthcare provider and for receiving a communication from the remote healthcare provider containing an instruction for use of the medical item, a specific therapy for the subject, or a medicine prescription.

11. An apparatus comprising:
a prescribed medical item for use as a therapy anticipated to be needed by a subject at a future point in time, the medical item selected based on a statistical level of likelihood that the therapy will be needed at the future point of time using one or more of a present biometric data about the subject or an environmental factor, wherein the dispensed medial item is a medicine or a non-medicine medical item;
a label or a token directly or indirectly attached to and detachable from the medical item, wherein the label or the token includes an information portion for providing information at least identifying the dispensed medical item, instructions for future use of the dispensed medical item, the subject, a medication data, or a combination thereof, and wherein the label or the token is electronically indexed to an electronic record stored in a remote database; and
means for obtaining the information from the label or token and for dispensing by a remote healthcare provider the medical item to the subject ahead of the need for the medical item by the subject, instructions for future use of the medical item, and monitoring a use of the medical item by the subject after dispensing.

12. The apparatus of claim 11, further comprising a communications device for receiving a signal generated by the remote healthcare provider.

13. The apparatus of claim 11, wherein the statistical level of likelihood is calculated based on one or more of the biometric data, a name, an age, a gender, and a physical position of the subject in space.

14. The apparatus of claim 11, wherein the information provided by the label or the token is electronically readable by an electronic device that transmits the information to a remote server.

15. The apparatus of claim 11, wherein the anticipated future need is based on a statistical level of likelihood that the items will be needed within a period of 24 months.

16. The system of claim 11, wherein the software is further adapted to causing the storing in at least the electronic record of the cumulative count of the physical tap or the use of the item.

17. A system comprising:
a storage container for providing to a subject and dispensing one or more prescribed medical items contained therein to the subject at a future point in time that is between 1 day and 24 months in the future;
a communications device for communicating information between a technology device and the storage container; and
a processor-executable software for enabling the communication of the information between the container and the technology device,
wherein the one or more medical items is selected based on a health or economic risk assessment, an output from a biometric sensor device, or a computed statistical level of likelihood that the one or more medical items will be a needed new therapy for the subject.

18. The system of claim 17, wherein the statistical level of likelihood that the one or more medical items will be needed at a future point of time is based on one or more of a biometric data about the subject or an environmental factor.
